SEQUENCE LISTING (42 sequences)

SEQ ID NO: 1 - RNA, 22 nt, Homo sapiens
uagcuuaucagacugauguuga

SEQ ID NO: 2 - DNA, 20 bp, Artificial Sequence
  GLI1_F_v1 primer
acaccggtaccactgtgtcc

SEQ ID NO: 3 - DNA, 20 bp, Artificial Sequence
  GLI1_R_v1 primer
cggctgacagtataggcaga

SEQ ID NO: 4 - DNA, 24 bp, Artificial Sequence
  GLI1_F_v2 primer
agtacatgctggtggttcacatgc

SEQ ID NO: 5 - DNA, 24 bp, Artificial Sequence
  GLI1_R_v2  primer
agtatgacttccggcacccttcaa

SEQ ID NO: 6 - DNA, 20 bp, Artificial Sequence
  SFRP1_F primer
tctgaggccatcattgaaca

SEQ ID NO: 7 - DNA, 20 bp, Artificial Sequence
  SFRP1_R primer
tcaggggcttcttcttcttg

SEQ ID NO: 8 - DNA, 21 bp, Artificial Sequence
  FOXA2_F primer
ccgactggagcagctactatg

SEQ ID NO: 9 - DNA, 20 bp, Artificial Sequence
  FOXA2_R primer
tgtacgtgttcatgccgttc

SEQ ID NO: 10 - DNA, 24 bp, Artificial Sequence
  AIM1_F primer
ctggaatgtcattatcagacacaa

SEQ ID NO: 11 - DNA, 20 bp, Artificial Sequence
  AIM1_R primer
tcagagacgtcgggttcact

SEQ ID NO: 12 - DNA, 18 bp, Artificial Sequence
  ALK_F primer
gggaagcatggttggaca

SEQ ID NO: 13 - DNA, 20 bp, Artificial Sequence
  ALK_R primer
actcgaaatgggttgtctgg

SEQ ID NO: 14 - DNA, 22 bp, Artificial Sequence
  CCDC62_F primer
aggaaacaaaaggcaacttcag

SEQ ID NO: 15 - DNA, 24 bp, Artificial Sequence
  CCDC62_R primer
attcattcagtattggtgtctgct

SEQ ID NO: 16 - DNA, 19 bp, Artificial Sequence
  CXCR7_F primer
cgatgcctccagagtctca

SEQ ID NO: 17 - DNA, 20 bp, Artificial Sequence
  CXCR7_R primer
ggcagatcatttggtgctct

SEQ ID NO: 18 - DNA, 20 bp, Artificial Sequence
  DIRAS2_F primer
gaagctctgagcggagttgt

SEQ ID NO: 19 - DNA, 19 bp, Artificial Sequence
  DIRAS2_R primer
tctgctcaggcatgttgct

SEQ ID NO: 20 - DNA, 18 bp, Artificial Sequence
  FAM101A_F primer
ctcagccgtaggcgtctt

SEQ ID NO: 21 - DNA, 20 bp, Artificial Sequence
  FAM101A_R primer
tgatgctctccccaaagaac

SEQ ID NO: 22 - DNA, 22 bp, Artificial Sequence
  GRK1_F primer
cccctcttcaaggaccttaact

SEQ ID NO: 23 - DNA, 20 bp, Artificial Sequence
  GRK1_R primer
ttggagtctgggatgaaagg

SEQ ID NO: 24 - DNA, 20 bp, Artificial Sequence
  HRASLS_F primer
agagaccccaggacacacac

SEQ ID NO: 25 - DNA, 21 bp, Artificial Sequence
  HRASLS_R primer
gcaatcattaaacgccatctc

SEQ ID NO: 26 - DNA, 19 bp, Artificial Sequence
  PPP2R2B_F primer
atcctgccaccatcacaac

SEQ ID NO: 27 - DNA, 20 bp, Artificial Sequence
  PPP2R2B_R primer
gcgttggcaaatactcttcg

SEQ ID NO: 28 - DNA, 20 bp, Artificial Sequence
  RGS20_F primer
cctcagggctgttcctgata

SEQ ID NO: 29 - DNA, 19 bp, Artificial Sequence
  RGS20_R primer
gctagaaaggaggccgaac

SEQ ID NO: 30 - DNA, 19 bp, Artificial Sequence
  SOX15_F primer
aagttcctcggcaacgact

SEQ ID NO: 31 - DNA, 20 bp, Artificial Sequence
  SOX15_R primer
gaagcgtcgatcctgaaaat

SEQ ID NO: 32 - DNA, 106 bp, Artificial Sequence
  partial human gene sequence after amplification of AIM1
ctggaatgtcattatcagacacaatgacacttagaggaagtgtccaaaataaactcaatccccgacctggaaaggtagtgatatatagtgaacccgacgtctctga

SEQ ID NO: 33 - DNA, 62 bp, Artificial Sequence
  partial human gene sequence after amplification of ALK
gggaagcatggttggacagtgctccagggaagaatcgggcgtccagacaacccatttcgagt

SEQ ID NO: 34 - DNA, 90 bp, Artificial Sequence
  partial human gene sequence after amplification of CCDC
aggaaacaaaaggcaacttcagtattcatcgtgatcacgaatttctcatctatgtggaaggcagaaagcagacaccaatactgaatgaat

SEQ ID NO: 35 - DNA, 64 bp, Artificial Sequence
  partial human gene sequence after amplification of CXCR7
cgatgcctccagagtctcagagacggagtactctgccttggagcagagcaccaaatgatctgcc

SEQ ID NO: 36 - DNA, 81 bp, Artificial Sequence
  partial human gene sequence after amplification of DIRAS2
gaagctctgagcggagttgtgttcttccccaggtgcgtcctggctgagagttggagctctccagcaacatgcctgagcaga

SEQ ID NO: 37 - DNA, 113 bp, Artificial Sequence
  partial human gene sequence after amplification of FAM101A
ctcagccgtaggcgtctttgcccggagctgtgagcccccctcccaactcccaaatcccccggcgtcggagatgaggccccggatgctgccagtgttctttggggagagcatca

SEQ ID NO: 38 - DNA, 74 bp, Artificial Sequence
  partial human gene sequence after amplification of GRK1
cccctcttcaaggaccttaactggaggcagctggaggctgggatgctgatgccccctttcatcccagactccaa

SEQ ID NO: 39 - DNA, 82 bp, Artificial Sequence
  partial human gene sequence after amplification of HRASLS
agagaccccaggacacacacagctgcctcccggtgcgagaagaagaccccggcttgagagtgagatggcgtttaatgattgc

SEQ ID NO: 40 - DNA, 91 bp, Artificial Sequence
  partial human gene sequence after amplification of PPP2R2B
atcctgccaccatcacaaccctgcgggtgcctgtcctgagacccatggacctgatggtggaggccaccccacgaagagtatttgccaacgc

SEQ ID NO: 41 - DNA, 91 bp, Artificial Sequence
  partial human gene sequence after amplification of RGS20
cctcagggctgttcctgatatcaagtccttcccgcctgcacagctcccagactcgcccgccgccccgaagctgttcggcctcctttctagc

SEQ ID NO: 42 - DNA, 60 bp, Artificial Sequence
  partial human gene sequence after amplification of SOX15
aagttcctcggcaacgactccagactgggaagacctttccattttcaggatcgacgcttc

(19) United States
(12) Patent Application Publication
(10) Pub. No.: US 2012/0245044 A1
(43) Pub. Date: Sep. 27, 2012

(54) METHODS OF DETERMINING CHEMOTHERAPY RESPONSE IN CANCER

(75) Inventor: Glen J. Weiss, Phoenix, AZ (US)

(73) Assignee: TRANSLATIONAL GENOMICS RESEARCH INSTITUTE, Phoenix, AZ (US)

(21) Appl. No.: 13/430,609

(22) Filed: Mar. 26, 2012

Related U.S. Application Data
(60) Provisional application No. 61/467,762, filed on Mar. 25, 2011.

(51) Int. Cl. C12Q 1/68; C12N 9/16; C40B 30/00; G01N 33/566; C07K 14/47; C07H 21/04

(52) U.S. Cl. 506/7; 530/350; 536/23.5; 435/6.12; 436/501; 435/7.1; 435/7.92; 435/196

(57) ABSTRACT

This disclosure provides a biomarker profile, which is linked to cancer cell chemo-resistance. The disclosure further provides methods of diagnosis and theranosis, and screening of new therapeutic agents using these biomarkers in the profile, and kits for employing these methods and compositions.

### CROSS REFERENCE

This application is related to and claims the priority benefit of U.S. provisional application 61/467,762, filed on Mar. 25, 2011, the teachings and content of which are incorporated by reference herein.

### SEQUENCE LISTING

This application contains a sequence listing in computer readable format, the teachings and content of which are hereby incorporated by reference.

### FIELD OF THE INVENTION

This invention relates to biomarkers indicating drug resistance to chemotherapeutics. Further, it relates to methods of using these biomarkers in screens and analyses, including prognoses, theranoses, and systems for identification of resistance of tumors to chemotherapy.

### BACKGROUND OF THE INVENTION

Cisplatin combination chemotherapy is the cornerstone of treatment of many cancers. Cisplatin (also called cisplatinum, or cis-diamminedichloroplatinum(II), or CDDP, and with trade names such as Platinol and Platinol-AQ) is used to treat various types of cancers, including sarcomas, some carcinomas (e.g., small cell lung cancer, and ovarian cancer), lymphomas, and germ cell tumors. It was the first member of a class of platinum-containing anti-cancer drugs, which now also includes carboplatin and oxaliplatin. Despite the fact that the initial platinum responsiveness is high, the majority of cancer patients, however, will eventually relapse with cisplatin-resistant disease. Many mechanisms of cisplatin resistance have been proposed including changes in cellular uptake and efflux of the drug, increased detoxification of the drug, inhibition of apoptosis and increased DNA repair, but there is still no clear understanding of cisplatin resistance in cancer patients.

Drug resistance to chemotherapeutics is particularly impairing in the treatment of small-cell lung cancer (SCLC). SCLC accounts for approximately 15% of all lung cancers, and is the most aggressive form of lung cancer. There are limited treatment options for this type of cancer, and cisplatin is the standard first-line SCLC chemotherapeutic drug. However, about 15-30% of SCLC patients are at high risk for cisplatin resistance. Further cisplatin is very toxic to SCLC patients, so much so that fewer than 40% of patients treated with cisplatin are in good enough condition to receive any additional therapy. Therefore, there is a need to determine or predict chemotherapy response in cancer patients, so that the patients can be placed on other effective treatments to avoid suffering unnecessary treatment side effects and cost, and missing a potential window of meaningful alternative treatment.

### REFERENCE TO COLOR FIGURES

This application file contains at least one photograph executed in color. Copies of this patent application publication with color photographs will be provided by the Office upon request and payment of the necessary fee.

### SUMMARY OF THE INVENTION

One aspect of the present invention provides a biomarker profile, which comprises one or more nucleic acids chosen from GLI1, SFRP1, FOXA2, MIR21, PPP2R2B and SOX15.

Another aspect of the present invention provides a method for identifying chemotherapeutic drug resistance in a subject. The method comprises (a) receiving the sample; and (b) detecting overexpression of one or more biomarkers selected from the group consisting of GLI1, SFRP1, FOXA2, MIR21, PPP2R2B and SOX15 in the sample relative to their respective control level of expression in a cancer cell responsive to the chemotherapeutic drug.

Yet another aspect of the present invention provides a method for identifying an agent that inhibits a therapeutic target. The method comprises (a) contacting a cancer cell with the agent, wherein the sample cancer cell comprises an over-expressed therapeutic target selected from the group consisting of GLI1, SFRP1, FOXA2, MIR21, PPP2R2B and SOX15; and (b) testing one or more cancer cell responses to the agent, wherein the cancer cell response is chosen from reaction to a chemotherapeutic drug, cancer cell count, metastasis, and apoptosis; wherein the cancer cell response is compared relative to a control.

Yet another aspect of the present invention provides a kit for detecting altered expression of one or more biomarkers associated with cancer cell resistance to a chemotherapeutic drug. The kit comprises one or more reagents for detecting at least one biomarker selected from the group consisting of GLI1, SFRP1, FOXA2, MIR21, PPP2R2B and SOX15.

Other aspects and iterations of the invention are described in more detail below.

### BRIEF DESCRIPTION OF FIGURES

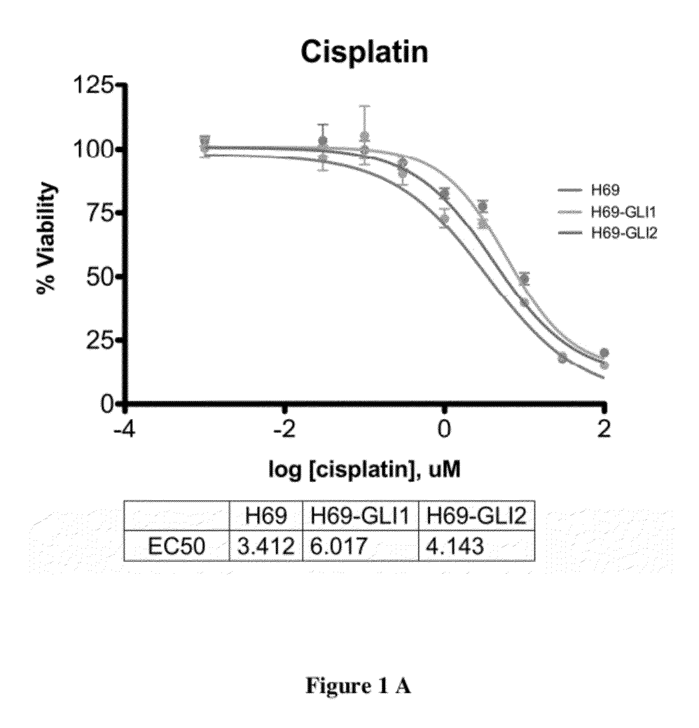

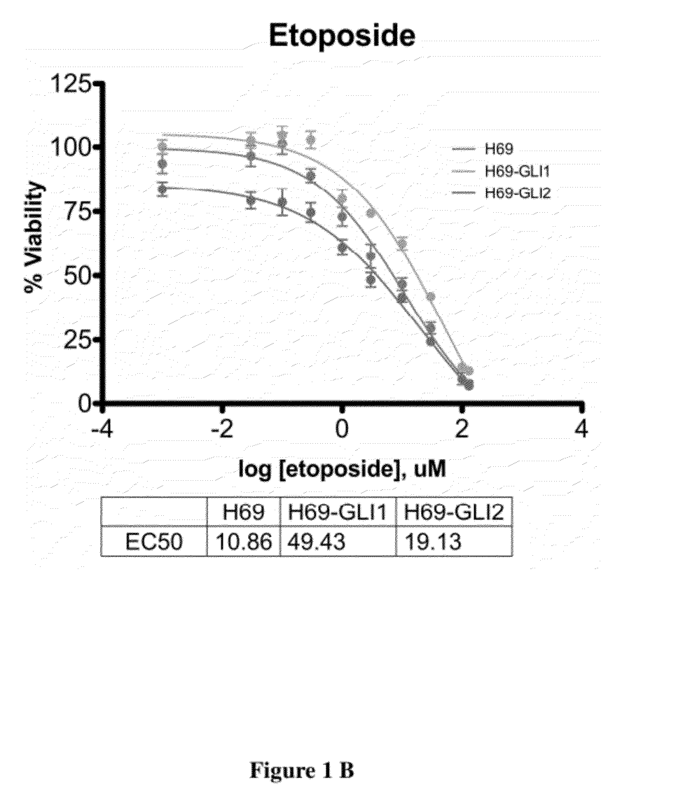

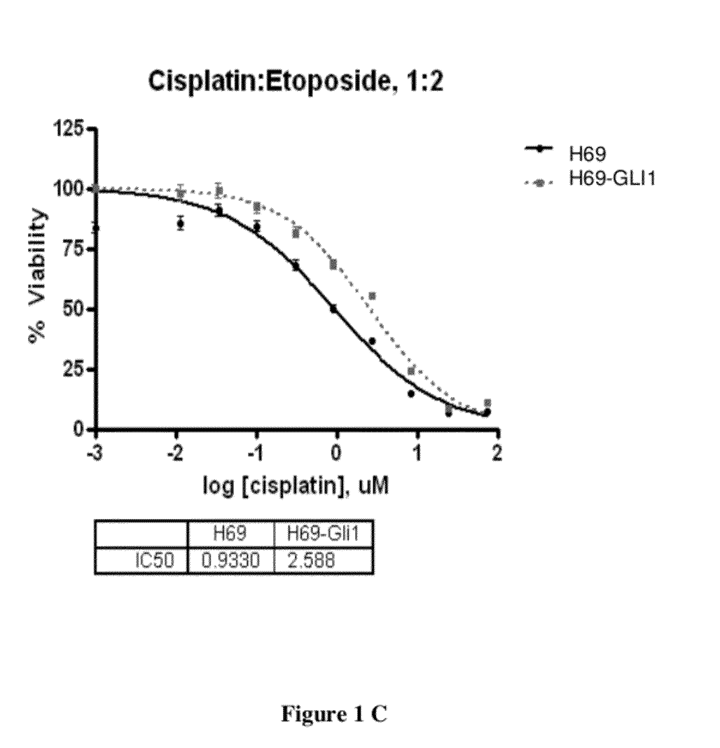

FIG. 1 depicts series of drug-dose response experiments for cisplatin, etoposide, and combination cisplatin/etoposide at 1:2 molar ratio: (A) Drug-dose response experiment in H69-GLI1 and H69-GLI2 versus H69-wt for cisplatin. (B) Drug-dose response experiment in H69-GLI1 and H69-GLI2 versus H69-wt for etoposide. (C) Drug-dose response experiment in H69-GLI1 versus H69-wt for cisplatin.

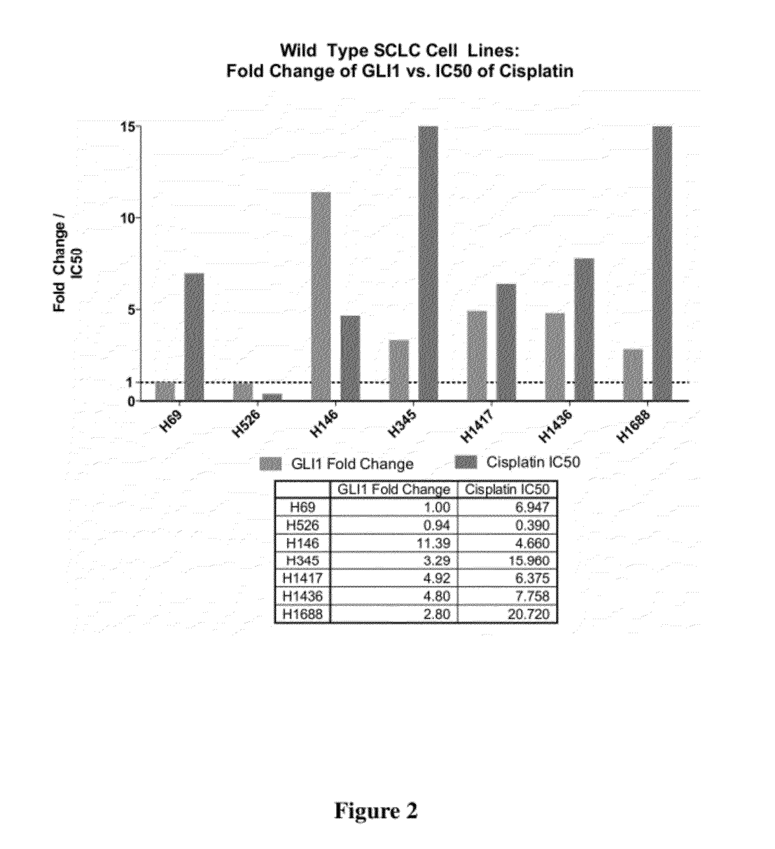

FIG. 2 depicts seven wild-type SCLC cell lines showing correlation between GLI1 expression (relative to H69-wt) and cisplation resistance, by IC50 from drug-dose response experiments.

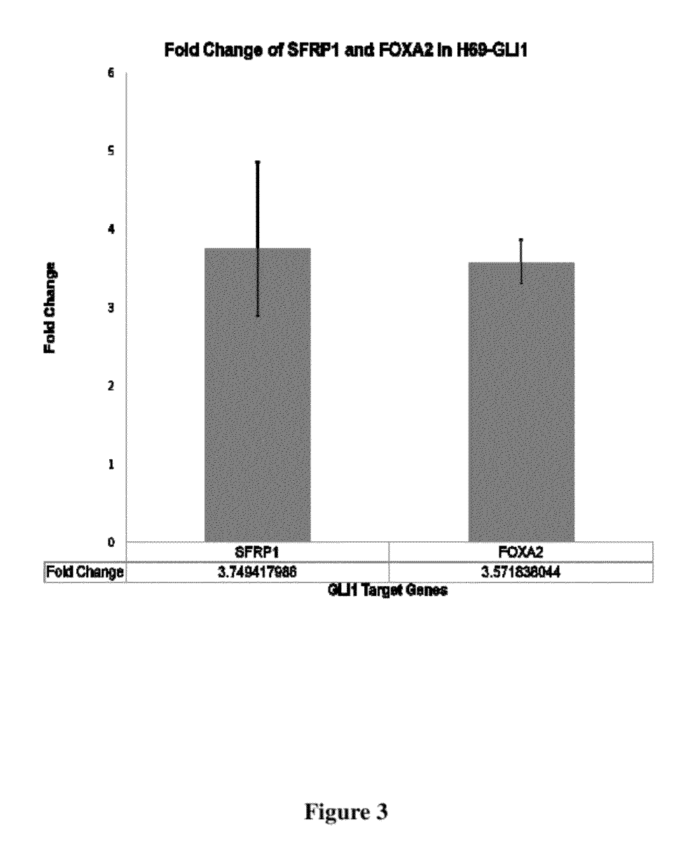

FIG. 3 depicts the qRT-PCR fold change analysis for SFRP1 and FOXA2. Fold change values for H69-GLI1 were normalized to H69-wt. mRNA expression values for SFRP1 and FOXA2 were normalized to GAPDH.

### DETAILED DESCRIPTION OF THE INVENTION

The present invention provides a set of biomarkers indicative of chemoresistance. Specifically, these biomarkers are GLI1, SFRP1, FOXA2, MIR21, PPP2R2B and SOX15. The invention also provides a method of identifying candidates for treatment with chemotherapy using these biomarkers, and a method for screening inhibitor to therapeutic targets such as GLI1, SFRP1, FOXA2, MIR21, PPP2R2B or SOX15.

I. Genes with Disrupted Expression in Cancer Cells

It is known that the gene expression patterns are complexly different between normal cells, cancer cells and chemo-resistant cancer cells. This invention discloses that in Small Cell Lung Cancer (SCLC) cells GLI1, SFRP1, FOXA2, MIR21, PPP2R2B and SOX15 genes are up-regulated, and the over-expression of these genes is associated with resistance to chemotherapeutic drugs, which include but are not limited to cisplatin and etoposide.

Glioma-associated oncogene family zinc finger 1 (GLI, UniProtKB/Swiss-Prot Accession No: P08151) is encoded by the GLI1 gene. GLI1 protein is a member of the Kruppel family of zinc finger proteins and it functions as a transcription factor. GLI1, as a transcription factor, is involved with regulation of a number of key genes in cell fate determination and proliferation. GLI may regulate the transcription of specific genes during normal craniofacial development and digital development, as well as development of the central nervous system and gastrointestinal tract. GLI also mediates cell proliferation and differentiation. GLI1's role in oncogenesis and cancer progression remains to be determined. Aberrant expression of a subset of hedgehog pathway related genes, including GLI1, was found in platinum-resistant SCLC cell lines. Multiple transcript variants encoding different isoforms of GLI1 have been found.

Secreted frizzled-related protein 1 (SFRP1, UniProtKB/Swiss-Prot Accession No: Q8N474) is encoded by the SFRP1 gene. SFRP1 contains a cysteine-rich domain homologous to the putative Wnt-binding site of Frizzled proteins. They have a role in regulating cell growth and differentiation in specific cell types. Members of this family act as soluble modulators of Wnt signaling. SFRP1 proteins function as modulators of Wnt signaling through direct interaction with Wnts. It is known that SFRP inhibits WNT1/WNT4-mediated TCF-dependent transcription. Epigenetic silencing of SFRP genes leads to deregulated activation of the Wnt-pathway which is associated with cancer. SFRP1 gene may also be involved in determining the polarity of photoreceptor cells in the retina. However SFRP1's role in platinum resistance in cancer has not been reported.

Forkhead box A2 (FOX2, UniProtKB/Swiss-Prot Accession No:Q9Y261) is encoded by the FOXA2 gene. The FOXA2 gene encodes a member of the forkhead class of DNA-binding proteins, which are transcription factors involved in embryonic development, establishment of tissue-specific gene expression and regulation of gene expression in differentiated tissues. Transcript variants encoding different isoforms have been identified for this gene. It is believed that FOXA2 is involved in the development of multiple endoderm-derived organ systems such as the liver, pancreas and lungs. More specifically, FOXA2 are hepatocyte nuclear factors activating liver-specific genes such as albumin and transthyretin. Similar family members in mice have roles in the regulation of metabolism and in the differentiation of the pancreas and liver. FOXA2 also interacts with chromatin. It may play a role in opening the compacted chromatin for other proteins through interactions with nucleosomal core histones, thereby replacing linker histones at target enhancer and/or promoter sites. FOXA2 was also found to function in glucose homeostasis, fat metabolism. However, FOXA2's role in platinum resistance in cancer was unknown.

Serine/threonine-protein phosphatase 2A 55 kDa regulatory subunit B beta isoform (PPP2R2B, UniProtKB/Swiss-Prot Accession No:Q00005) is encoded by the PPP2R2B gene. PPP2R2B encodes the B regulatory subunit that associates with the common heterodimeric core enzyme of PP2A (Serine/threonine-protein phosphatase 2A) consisting of a 36 kDa catalytic subunit (subunit C) and a 65 kDa constant regulatory subunit (PR65 or subunit A). The B regulatory subunit is likely involved in modulating substrate selectivity and catalytic activity, and may also direct the localization of the catalytic enzyme to a particular subcellular compartment. Within the PP2A holoenzyme complex, PPP2R2B is required to promote proapoptotic activity. In addition, PPP2R2B may regulate neuronal survival through the mitochondrial fission and fusion balance.

Protein SOX15 (SOX15, UniProtKB/Swiss-Prot Accession No:060248) is encoded by SOX15 gene. SOX15 is a nucleus protein containing one HMG box DNA-binding domain and SOX15 binds to the 5′-AACAAT-3′ sequence. SOX15 may function as a transcription factor regulating target gene expression through specific binding.

It has been observed that miRNAs frequently are located at chromosomal regions deleted or amplified in cancers, suggesting that miRNAs are a class of genes involved in human tumorigenesis. MicroRNAs (miRNAs) are small, non-coding RNA molecules of about 18 to 25 nucleotides that are involved in post-transcriptional regulation of gene expression in multicellular organisms by affecting both the stability and translation of mRNAs. miRNAs are transcribed by RNA polymerase II, and the primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA (miRNA*). The mature miRNA is incorporated into a RNA-induced silencing complex (RISC), which recognizes target mRNAs through imperfect base pairing with the miRNA and most commonly results in translational inhibition or destabilization of the target mRNA. By way of example, up-regulated MIR21 gene expression has previously been related to various cancers. However, its role in chemoresistance of cancer cells remains to be determined.

A higher expression level of the above genes in platinum resistant SCLC cells may result from these genes' allelic difference. Different expression may be because of various gene alleles in resistant and non-resistant cancer cells. An allele includes any form of a particular nucleic acid that may be recognized as a form of the particular nucleic acid on account of its location, sequence, expression level, expression specificity or any other characteristic that may identify it as being a form of the particular gene. Alleles includes, but need not be limited to, forms of a gene that include point mutations, silent mutations, deletions, frameshift mutations, single nucleotide polymorphisms (SNPs), inversions, translocations, heterochromatic insertions, and differentially methylated sequences relative to a reference gene, whether alone or in combination. An allele of a gene may or may not produce a functional protein; may produce a protein with altered function, localization, stability, dimerization, or protein-protein interaction; may have overexpression, underexpression or no expression; and/or, may have altered temporal or spacial expression specificity.

An allele may be compared to another allele that may be termed a wild type form of an allele. In comparison to the wild type allele, a different allele may be called a mutation or a mutant. Mutants may also be interchangeably called variants. In some cases, the wild type allele is more common than the mutant. A genetic mutation or variance may be any detectable change in genetic material such as DNA, or a corresponding change in the RNA or protein product of that genetic material. In the example of gene mutation, the DNA sequence of a gene or any controlling elements surrounding the gene is altered. Controlling elements include promoter, enhancer, suppressor or silencing elements capable of controlling a given gene. Other examples of mutations include alterations in the products of gene expression such as RNA or protein that result from corresponding mutations in the DNA.

Conserved variants encompass any mutation or other variant in which a given amino acid residue in a protein or enzyme has been changed without altering the overall conformation and function of the polypeptide, including, but not limited to, replacement of an amino acid with one having similar properties (for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Depending on the location of the variance in the overall context of the protein, some substitutions may have little or no effect on the apparent molecular weight or isoelectric point of the protein or polypeptide.

Amino acids other than those indicated as conserved may differ in a protein or enzyme so that the percent protein or amino acid sequence similarity between any two proteins of similar function may vary and may be, for example, from about 70% to about 99% as determined according to an alignment scheme such as by the Cluster Method, wherein similarity is based on the MEGALIGN algorithm. The concept of a variant further encompasses a polypeptide or enzyme which has at least 60%, 75%, 85%, 90%, or 95% amino acid identity as determined by algorithms, such as BLAST or FASTA, and which has the same or substantially similar properties and/or activities as the native or parent protein or enzyme to which it is compared.

Another example of gene variant is a gain-of-function variant. Gain-of-function variants of polypeptides encompass any variant in which a change in one or more amino acid residues in a protein or enzyme improves the activity of the polypeptide. Examples of activities of a polypeptide that may be improved by a change resulting in a gain of function variant include but are not limited to enzymatic activity, binding affinity, phosphorylation or dephosphorylation efficiency, activation, deactivation, or any other activity or property of a protein that may be quantitatively measured by some method now known or yet to be disclosed.

The presence or absence of an allele may be detected through the use of any process known in the art, including using primers and probes designed according to a specific allele for PCR, sequencing, hybridization analyses. In this invention, the chemoresistance related overexpression refers to the overexpression of the GLI1, SFRP1, FOXA2, MIR21, PPP2R2B and SOX15 genes and the alleles thereof, and the RNA or proteins of these genes and alleles thereof.

II. Biomarkers and Therapeutic Targets

Among the various aspects of the present disclosure is the provision of a set of biomarkers for chemoresistance. Specifically, the biomarkers herein are GLI1, SFRP1, FOXA2, MIR21, PPP2R2B and SOX15. As used herein, the term “biomarker” refers to a gene and its gene products (i.e., RNA and protein) whose expression is indicative of a particular phenotype or cellular condition, or physiological characteristic. As one exemplary example, when a gene is up-regulated in a particular cellular or physiological state, the over-expression of this gene and its RNA or protein product may be used as a biomarker that indicates an association with the particular cellular or physiological characteristic. As used herein “a biomarker profile” describes one or more biomarkers, each of which is associated with the same, or similar, phenotype or cellular condition, or physiological characteristic.

Prediction of a cellular or physiological characteristic may be achieved by assessing the expression of one or more biomarkers. Genes or their alleles with disrupted expression may be used as diagnostic, prognostic or theranostic biomarkers. One type of cellular or physiological characteristic of cancer is the response of the cancer cell to a particular treatment. In one exemplary example, the treatment is a chemotherapy. The cancer cell may be responsive, non-responsive or becoming resistant after being responsive to the treatment for a period of time. Assessing cancer response to a therapeutic drug includes the performing of any type of test, assay, or examination based on one or more biomarkers, from which the result, readout, or interpretation are correlated with an increased or decreased probability that a subject will be responsive or resistant to the therapy for a desired therapy outcome. Examples of therapy outcomes include, but need not be limited to survival, death, progression of existing disease, remission of existing disease, initiation of onset of a disease in an otherwise disease-free subject, or the continued lack of disease in a subject in which there has been a remission of disease. Assessing the risk of a disease outcome based on one or more biomarkers encompasses diagnosis in which the type of disease afflicting a subject is determined, specifically in this invention, a platinum-responsive type of cancer or a platinum-resistant type of cancer. Assessing therapy outcome of a particular disease based on one or more biomarkers encompasses the concept of prognosis. A prognosis may be any assessment of the risk of disease outcome in an individual in which a particular type of disease has been diagnosed. Assessing the risk based on one or more biomarkers further encompasses theranoses, that is, the prediction of therapeutic response in which a treatment regimen is chosen based on the assessment. Assessing the risk also encompasses a prediction of overall survival after diagnosis.

Some biomarkers may also be therapeutic targets for disease treatment. For example, inhibitors can be used to prevent the over-expression of a biomarker associated with a cellular or physiological characteristic to achieve therapeutic effects. In this sense, the over-expressed biomarker is a target for therapeutic purposes. Generally, a target may be any molecular structure produced by a cell, expressed inside the cell, accessible on the cell surface, or secreted by the cell. A target may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination. Specifically in this invention, a target may be represented by a nucleic acid sequence, the protein or peptide or the fragments thereof encoded by the nucleic acid sequence, such as GLI1, SFRP1, FOXA2, MIR21, PPP2R2B and SOX15. Examples of such nucleic acid sequences include miRNA, tRNA, siRNA, mRNA, cDNA, or genomic DNA sequences. Further, a target may be represented by a nucleic acid sequence in a form with or without epigenetic modifications. In addition, a target may be represented by a nucleic acid sequence in a form with SNPs (single nucleiotide polymorphism), point mutations, silent mutations, deletions, insertions, frameshift mutations, translocations, alternative splicing derivatives. Alternatively, a target may be represented by a protein or peptide or the fragments thereof with or without post-translational modifications.

In a preferred embodiment of the invention, the one or more biomarkers associated with chemo-resistance in a cancer cell is chosen from GLI1, SFRP1, FOXA2, MIR21, PPP2R2B and SOX15.

In another embodiment of the invention, the one or more targets for chemo-resistance cancer cell treatment is chosen from GLI1, SFRP1, FOXA2, MIR21, PPP2R2B and SOX15. In one preferred embodiment, the target for chemo-resistance cancer cell treatment is GLI1.

III. Methods of Identifying Candidates for Treatment with Chemotherapy Using Biomarkers

One aspect of this invention provides a method of identifying subjects for treatment with chemotherapy, and excluding subjects with chemo-resistance from chemotherapy using biomarkers. Specifically, the biomarkers disclosed in this invention include GLI1, SFRP1, FOXA2, MIR21, PPP2R2B and SOX15, of which the over-expression of each of these biomarkers is associated with chemo-resistance of the cancer cell.

The expression of a biomarker in a test sample may be more or less than that of a predetermined level to predict the presence or absence of a cellular or physiological characteristic. The expression of the biomarker or target in the test subject may be 1,000,000×, 100,000×, 10,000×, 1000×, 100×, 10×, 5×, 2×, 1×, 0.5×, 0.1×, 0.01×, 0.001×, 0.0001×, 0.00001×, 0.000001×, or 0.0000001× of the predetermined level indicting the presence or absence of a cellular or physiological characteristic. The predetermined level of expression may be derived from a single control sample or a set of control samples.

1. Subject and Samples

One aspect of the invention provides assessing the expression of one or more biomarkers in a biological sample from a subject. A subject includes any human or non-human mammal, including primate, cow, horse, pig, sheep, goat, dog, cat, or rodent. A subject, including a human patient, may be suspected of having a particular type of cancer, may have been diagnosed with a particular type of cancer, or may have a family history of a particular type of cancer. Methods of identifying subjects suspected of having cancer include but are not limited to: physical examination, family medical history, subject medical history, biopsy, or a number of imaging technologies such as ultrasonography, computed tomography, magnetic resonance imaging, magnetic resonance spectroscopy, or positron emission tomography.

Examples of sources of samples include but are not limited to biopsy or other in vivo or ex vivo analysis of prostate, breast, skin, muscle, facia, brain, endometrium, lung, head and neck, pancreas, small intestine, blood, liver, testes, ovaries, colon, stomach, esophagus, spleen, lymph node, bone marrow, kidney, placenta, or fetus. In some aspects of the invention, the sample may be a body fluid sample, such as peripheral blood, serum, plasma, lymph fluid, ascites, serous fluid, pleural effusion, sputum, cerebrospinal fluid, amniotic fluid, lacrimal fluid, gastric fluid, pancreatic fluid, mucus or urine, from which free floating DNA, RNA, protein, peptide or fragments thereof may be detected and compared to control samples. Samples include single cells, whole organs or any fraction of a whole organ, in any condition including in vitro, ex vivo, in vivo, post-mortem, fresh, fixed, or frozen. Alternatively, a sample may be any cell source from which DNA, including genomic, somatic, and germline DNA may be obtained.

The cell in a sample may be a tumor cell or a cancer cell. Tumor cells may be obtained by any method now known in the art or yet to be disclosed, including for example, surgical resection, laser capture microdissection, isolation from blood or other fluids including lavage fluid, or any other method capable of obtaining and, if necessary, concentrating tumor cells. These tumor cells include any cells derived from a tumor, neoplasm, cancer, precancer, cell line, malignancy, or any other source of cells that have the potential to expand and grow to an unlimited degree. Cancer cells may be derived from naturally occurring sources or may be artificially created cell lines. Cancer cells may also be capable of invasion into other tissues and metastasis when placed into an animal host. Cancer cells further encompass any malignant cells that have invaded other tissues and/or metastasized. One or more cancer cells in the context of an organism may also be called a cancer, tumor, neoplasm, growth, malignancy, or any other term used in the art to describe cells in a cancerous state.

Examples of cancers that could serve as sources of cancer cells include solid tumors such as fibrosarcoma, myxosarcoma, liposarcoma, chondrosarcoma, osteogenic sarcoma, chordoma, angiosarcoma, endotheliol sarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Additional cancers that may serve as sources of cancer cells include blood borne cancers such as acute lymphoblastic leukemia (“ALL,”), acute lymphoblastic B-cell leukemia, acute lymphoblastic T-cell leukemia, acute myeloblastic leukemia (“AML”), acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), hairy cell leukemia, multiple myeloma, lymphoblastic leukemia, myelogenous leukemia, lymphocytic leukemia, myelocytic leukemia, Hodgkin's disease, non-Hodgkin's Lymphoma, Waldenstrom's macroglobulinemia, Heavy chain disease, and Polycythemia vera.

2. Biomarker Expression Detection

The expression level of a biomarker can be determined, for example, by comparing mRNA or protein level in a test subject or sample to a control or a standard developed through accumulated data of controls or population samples. In one embodiment of this invention, the comparison is between the test subject or sample and the cisplatin-responsive subject or sample. In one embodiment, the expression of the biomarker in a sample may be compared to a control level of expression predetermined to predict the presence or absence of a particular physiological characteristic. The predetermined control level of biomarker expression may be derived from a single control or a set of controls. Alternatively, a control may be a sample having a previously determined control level of expression of a specific biomarker. Comparison of the expression of the biomarker in the sample to a control level of expression results in a prediction that the sample exhibits or does not exhibit the cellular or physiological characteristic.

Expression of a biomarker may be assessed by any number of methods used to detect material derived from a nucleic acid template used currently in the art, as well as those yet to be developed. Such methods may include any biomarker nucleic acid detection method, such as the following nonlimiting examples, microarray analysis, RNA in situ hybridization, RNAse protection assay, Northern blot, reverse transcriptase PCR, quantitative PCR, quantitative reverse transcriptase PCR, quantitative real-time reverse transcriptase PCR, and reverse transcriptase treatment followed by direct sequencing. Other examples include any method of assessing biomarker protein expression, such as, flow cytometry, immunohistochemistry, ELISA, Western blot, and immunoaffinity chromatography, HPLC, mass spectrometry, protein microarray analysis, PAGE analysis, isoelectric focusing, 2-D gel electrophoresis, or any enzymatic assay.

Other methods used to assess biomarker expression include the use of natural or artificial ligands capable of specifically binding a biomarker or a target. Such ligands include antibodies, antibody complexes, conjugates, natural ligands, small molecules, nanoparticles, or any other molecular entity capable of specific binding to a target. The term “antibody” is used herein in the broadest sense and refers generally to a molecule that contains at least one antigen binding site that immunospecifically binds to a particular antigen target of interest. Antibody, thus, includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term, thus, includes full length antibodies and/or their variants as well as immunologically active fragments thereof, thus encompassing, antibody fragments capable of binding to a biological molecule (such as an antigen or receptor) or portions thereof, including but not limited to Fab, Fab', F(ab′)2, facb, pFc', Fd, Fv or scFv (See, e.g., CURRENT PROTOCOLS IN IMMUNOLOGY, (Colligan et al., eds., John Wiley & Sons, Inc., NY, 1994-2001).

Ligands may be associated with a label, such as, a radioactive isotope or chelate thereof, dye (fluorescent or nonfluorescent) stain, enzyme, metal, or any other substance capable of aiding a machine or a human eye from differentiating a cell expressing a target from a cell not expressing a target. Additionally, expression may be assessed by monomeric or multimeric ligands associated with substances capable of killing the cell. Such substances include protein or small molecule toxins, cytokines, pro-apoptotic substances, pore forming substances, radioactive isotopes, or any other substance capable of killing a cell.

In addition, biomarker differential expression encompasses any detectable difference between the expression of a biomarker in one sample relative to the expression of the biomarker in another sample. Differential expression may be assessed by a detector, an instrument containing a detector, or by aided or unaided human eye. Examples include but are not limited to differential staining of cells in an IHC assay configured to detect a target, differential detection of bound RNA on a microarray to which a sequence capable of binding to the target is bound, differential results in measuring RT-PCR measured in ΔCt or alternatively inv the number of PCR cycles necessary to reach a particular optical density at a wavelength at which a double stranded DNA binding dye (e.g. SYBR Green) incorporates, differential results in measuring label from a reporter probe used in a real-time RT-PCR reaction, differential detection of fluorescence on cells using a flow cytometer, differential intensities of bands in a Northern blot, differential intensities of bands in an RNAse protection assay, differential cell death measured by apoptotic markers, differential cell death measured by shrinkage of a tumor, or any method that allows the detection of a difference in signal between one sample or set of samples and another sample or set of samples.

Techniques using microarrays may also be advantageously implemented to detect genetic abnormalities or assess gene expression. Gene expression may be that of the one or more biomarkers chosen from GLI1, SFRP1, FOXA2, MIR21, PPP2R2B and SOX15 or the expression of another set of genes upstream or downstream in a pathway of which the one or more biomarkers is a component or a regulator. In one embodiment, microarrays may be designed so that the same set of identical oligonucleotides is attached to at least two selected discrete regions of the array, so that one can easily compare a normal sample, contacted with one of said selected regions of the array, against a test sample, contacted with another of said selected regions. Examples of microarray techniques include those developed by Nanogen, Inc. (San Diego, Calif.) and those developed by Affymetrix (Santa Clara, Calif.). However, all types of microarrays, also called “gene chips” or “DNA chips”, may be adapted for the identification of mutations. Such microarrays are well known in the art.

In one embodiment of detecting the presence of a biomarker associated with a characteristic of a disease, a threshold value may be obtained by performing the one or more above mentioned assays on samples obtained from a population of patients having a certain disease condition (chemo-resistant cancer, for example) and from a second population of subjects that do not have the disease condition. In assessing disease outcome or the effect of treatment, a population of patients with a disease condition may be followed for a period of time. After the period of time expires, the population may be divided into two or more groups based on one or more parameters. For example, the population may be divided into a first group of patients whose disease progresses to a particular endpoint and a second group of patients whose disease does not progress to the particular endpoint. Examples of endpoints include disease recurrence, death, metastasis, chemotherapy response or resistance, or other clinically meaningful indexes. Based on the observation of the parameters, a predetermined level of expression of a biomarker for each group may be selected to signify a particular physiological or cellular characteristic including identifying or diagnosing a particular disease, assessing a risk of outcome or a prognostic risk, or assessing the risk that a particular treatment will or will not be effective. If expression of the biomarker in a test sample is more similar to the predetermined expression of the biomarker in one group relative to the other group, the sample may be assigned a risk of having the same outcome as the patient group to which it is more similar.

Additionally, a predetermined level of biomarker expression may be established by assessing the expression of a biomarker in a sample obtained first from one patient, assessing the expression of the biomarker in additional samples obtained later in time from the same patient, and comparing the expression of the biomarker from the samples later in time with the previous sample(s). This method may be used in the case of a biomarker that indicates, for example, progression of disease, lack of efficacy of a treatment regimen, remission of a disease, or efficacy of a treatment regimen.

In a preferred embodiment, predicting a test sample or subject's response to a therapy, such as a drug therapy, is based on the detection of an altered expression of a biomarker in the test subject or sample in comparison to the expression level in a subject or sample responsive to the therapy, and the one or more biomarkers is selected from GLI1, SFRP1, FOXA2, MIR21, PPP2R2B and SOX15. In preferred forms, the subject's response will determine the selection of appropriate interventional therapies.

IV. Method for Screening Inhibitor to Therapeutic Targets

Disclosed herein are methods for identifying agents that inhibit overexpression of therapeutic targets, including GLI1, SFRP1, FOXA2, MIR21, PPP2R2B and SOX15, in cancer cells, and specifically, in chemoresistant cancer cells. Preferably, the target for a therapeutic agent is GLI1. The methods include contacting an inhibiting agent with a cell comprising a therapeutic target that is over-expressed, the target being GLI1, SFRP1, FOXA2, MIR21, PPP2R2B and SOX15. An agent that is an inhibitor of cancer condition may be identified by determining the effect of the agent on the expression level of a target. In an example, an agent that down-regulates the target expression as compared to the target expression in the absence of the agent identifies that agent as an inhibitor of a target; and specifically, in the present invention, the target is for chemoresistance and the agent is an inhibitor to this chemoresistance target.

Inhibitors of chemoresistant gene or protein expression may be any agent including, for example, a pharmaceutically active ingredient or pharmaceutically acceptable salt thereof, a drug, a toxin, a chemical, a small organic molecule, a large molecule, peptide, or an antibody. Large-molecule pharmaceuticals refer to pharmaceutical agents having a molecular weight greater than about 1000 daltons, e.g., peptidic drugs, vaccines and hormones. The term “antibody” used herein is similarly defined as in Section III. 2.

The screening or creation, identification and selection of appropriate inhibitors of chemoresistant targets described herein can be accomplished by a variety of methods. One approach is to use structural knowledge about the target protein to design a candidate molecule with which it will precisely interact, an example of which would be computer assisted molecular design. A second approach is to use combinatorial or other libraries of molecules, whereby a large library of molecules is screened for inhibitory effect with regard to the target gene or protein expression, or ability to inhibit the transcriptional factor activity of the target protein. In a further example, a panel of antibodies may be screened for the ability to inhibit the target protein.

Cancer and precancer may be thought of as diseases that involve unregulated cell growth. Metastasis involves migration of tumor cells away from the site of the primary tumor, entry into the circulation, and proliferation at a new site. Cell growth involves a number of different factors. One factor is how rapidly cells proliferate, and another involves how rapidly cells die. Cells can die either by necrosis or apoptosis depending on the type of environmental stimuli. Cell motility is yet another factor that influences tumor growth kinetics and metastasis. Resolving which of the many aspects of cell growth an agent affects can be important to the discovery of a relevant pharmaceutical therapy for chemoresistant cancer cells. Screening assays based on this technology can be combined with other tests to determine which agents have growth inhibiting and pro-apoptotic activity in chemoresistant cancer cells under the treatment of a chemotherapeutic drug, such as, cisplatin.

Some embodiments provided herein involve determining the ability of a given agent to inhibit the overexpression of GLI1, SFRP1, FOXA2, MIR21, PPP2R2B or SOX15. In one preferred embodiment, the target is GLI1. Agents can be assessed for their probable ability to treat neoplastic lesions, either directly or indirectly, in chemoresistant cancer cells that are treated with a chemotherapeutic drug, such as cisplatin. Various cell lines can be used, which may be selected based on the tissue to be tested. Certain cell lines are well characterized and are used, for instance, by the United States National Cancer Institute (NCI) in their screening program for new anti-cancer drugs. Cell lines can also be constructed to overexpress GLI1, SFRP1, FOXA2, MIR21, PPP2R2B or SOX15 for screening inhibitory agents for cancer cells, or specifically chemoresistant cancer cells. Significant tumor cell growth inhibition, greater than about 30% at a dose of 100 μM or below, is further indicative that the agent is useful for treating neoplastic lesions. An IC₅₀ value may also be determined and used for comparative purposes. This value is the concentration of drug needed to inhibit tumor cell growth by 50% relative to the control. In some embodiments, the IC₅₀ value is less than 100 μM in order for the agent to be considered further for potential use for treating, ameliorating, or preventing neoplastic lesions or tumor metastasis.

In another embodiment, agents can be screened for induction of apoptosis, or cell death, using cultures of chemoresistant tumor cells in the presence of a chemotherapuetic drug, such as cisplatin, using GLI1, SFRP1, FOXA2, MIR21, PPP2R2B or SOX15 as a target. In a preferred embodiment, the target is GLI1. In some examples of such screening methods, treatment of cells with agents involves either pre- or post-confluent cultures and treatment for 1 to 7 days at various concentrations of the agents. Apoptotic cells can be measured in both the attached and “floating” portions of the cultures. Both portions are collected by removing the supernatant, trypsinizing the attached cells, and combining both preparations following a centrifugation wash step (for example, 10 minutes, 2000 rpm). Following treatment with an agent, cultures can be assayed for apoptosis and necrosis, for instance, by florescent microscopy following labeling with acridine orange and ethidium bromide. Many methods for measuring apoptotic cells are known to those of ordinary skill in the art; for instance, one method for measuring apoptotic cell number has been described by Duke & Cohen (Curr. Prot. Immuno., Coligan et al., eds., 3.17.1-3.17.1, 1992).

Apoptosis may also be quantified by measuring an increase in DNA fragmentation in cells that have been treated with agents. Commercial photometric enzyme immunoassays (EIA) for the quantitative in vitro determination of cytoplasmic histone-associated-DNA-fragments (mono- and oligo-nucleosomes) are available (e.g., Cell Death Detection ELISA, Boehringer Mannheim). The cell death detection assay is based on a sandwich-enzyme-immunoassay principle, using mouse monoclonal antibodies directed against DNA and histones, respectively. This assay allows the specific determination of mono- and oligo-nucleosomes in the cytoplasmic fraction of cell lysates.

Statistically significant increases of apoptosis (i.e., greater than 2 fold stimulation at an agent concentration of 100 μM) are further indicative that the agent is useful for treating neoplastic lesions. Preferably, the EC₅₀ value for apoptotic activity should be less than 100 μM for the agent to be further considered for potential use for treating neoplastic lesions. EC₅₀ is understood herein to be the concentration that causes 50% induction of apoptosis relative to vehicle (control) treatment.

In another embodiment, agents can be screened for inhibitory effects to the activity of GLI1, SFRP1, FOXA2, MIR21, PPP2R2B or SOX15 as a transcription regulator of their respective down stream genes. In one preferred embodiment, the screening of inhibitory agents is achieved through determining the expression or activity of a downstream gene or protein known to be specifically regulated by GLI1.

V. Kits

The invention further provides kits that facilitate the detection of altered expression of one or more biomarkers associated with drug resistance to chemotherapy. The kit may comprise one or more reagents used to include or exclude a patient from a chemotherapy treatment. The reagents in the kit may be primers, probes, and/or antibodies that are capable of identifying a biomarker or target associated to drug resistance to chemotherapy selected from the group consisting of GLI1, SFRP1, FOXA2, MIR21, PPP2R2B and SOX15.

A kit that facilitates nucleic acid based assays may further comprise one or more of the following: nucleic acid extraction reagents, controls, disposable cartridges, labeling reagents, enzymes including PCR amplification reagents such as the DNA polymerases Taq or Pfu, reverse transcriptase, or one or more other polymerases, and/or reagents that facilitate hybridization.

The kit may further comprise a label that can be used to label the primer or probe oligonucleotide. A label may be any substance capable of aiding a machine, detector, sensor, device, or enhanced or unenhanced human eye from differentiating a sample that that displays increased expression from a sample that displays reduced expression. Examples of labels include but are not limited to: a radioactive isotope or chelate thereof, a dye (fluorescent or nonfluorescent,) stain, enzyme, or nonradioactive metal. Specific examples of labels include, but are not limited to: fluorescein, biotin, digoxigenin, alkaline phosphatase, biotin, streptavidin, ³H, ¹⁴C, ³²P, ³⁵S, or any other compound capable of emitting radiation, rhodamine, 4-(4′-dimethylaminophenylazo) benzoic acid (“Dabcyl”); 4-(4′-dimethylamino-phenylazo)sulfonic acid (sulfonyl chloride) (“Dabsyl”); 5-((2-aminoethyl)-amino)-naphtalene-1-sulfonic acid (“EDANS”); Psoralene derivatives, haptens, cyanines, acridines, fluorescent rhodol derivatives, cholesterol derivatives; ethylene diamine tetra-acetic acid (“EDTA”) and derivatives thereof, or any other compound that signals the presence of the labeled nucleic acid. In one embodiment of the invention, the label includes one or more dyes optimized for use in genotyping. Examples of such dyes include, but are not limited to: dR110, 5-FAM, 6FAM, dR6G, JOE, HEX, VIC, TET, dTAMRA, TAMRA, NED, dROX, PET, BHQ+, Gold540, and LIZ.

In yet anther embodiment, the primers and probes in the kit may have been labeled, and can be applied without labeling process in PCR, sequencing reaction, or binding to a solid substrate such as oligonucleotide array.

The kit that facilitates the detection of altered expression of one or more biomarkers or targets associated to drug resistance to chemotherapy may also comprise instructions for use. In one embodiment, the kit may further comprise an indication that links the output of the assays provided by the kit to a particular result. For example, an indication may provide guidance to associate the presence or absence of one or more sequences to a specific treatment plan. The output of the assay may be in a form of a particular sequence, a particular genotype, a particular expression level in a real-time quantitative PCR reaction, a level of fluorescence or radioactive decay, a value derived from a standard curve, a positive or negative control, or any combination of these and other outputs. The indication may be printed on a writing that may be included in the kit or it may be posted on the Internet or embedded in a software package. The writing may include graphical depictions of results such as a photomicrograph or amplification plot.

The kit that facilitates the detection of altered expression of one or more biomarkers or targets associated to drug resistance to chemotherapy may further comprise a device used to collect the sample. Such devices may include, but need not be limited to: swabs, needles, blood collection tubes, wipes, or any other apparatus that may be used to collect a biological sample from a subject.

### EXAMPLE

The following non-limiting example is intended to further illustrate and explain the present invention. The invention, however, should not be limited to any of the details in the example.

### Example 1

### Increased GLI1 Expression Correlated with Increased Cisplatin Resistance

Methods. Two SCLC cell lines, H69 and H526, were transduced with a GLI1 over-expressing (o/e) vector (Kasper et al, 2007) to create two lines expressing the gene: H69-GLI1 and H526-GLI1. GLI1 expression was verified by qRT-PCR. Cellular proliferation was measured over a 96-hour period. To study the drug dose response, GLI1 o/e and parental lines were treated with single-agent or combination dosing of cisplatin and etoposide, and cell viability was assessed after 72 hours. Relative GLI1 expression was compared with cisplatin response in four additional SCLC cell lines (H146, H187, H345, H1688). Gene expression analysis was performed on H69-GLI1 and H69 parental cells, with two replicates for each, using Agilent (Agilent Technologies, Santa Clara, Calif.) 44K expression arrays. Data was normalized and analyzed using Genespring software (Agilent Technologies, Santa Clara, Calif.). Changes greater than 2 fold were considered significant.

Results. Compared to parental cells, GLI1 expression was 11 fold and 149 fold higher in H69-GLI1 and H526-GLI1, respectively. Proliferation assays showed no significant differences H69-wt vs. H69-GLI1 cells. Drug dose response experiments showed at least a 1.5 fold shift in cisplatin resistance in H69-GLI1 compared to H69-wt (FIG. 1). Series of drug-dose response experiments for cisplatin, etoposide, and combination cisplatin/etoposide at 1:2 molar ratio were performed. The fold change between H69-GLI1 and H69-wt was 1.76 and fold change between H69-GLI2 and H69-wt was 1.21 in the drug-dose response experiment in H69-GLI1 and H69-GLI2 versus H69-wt for cisplatin. The fold change between H69-GLI1 and H69-wt was 4.55 and fold change between H69-GLI2 and H69-wt was 1.76 in the drug-dose response experiment in H69-GLI1 and H69-GLI2 versus H69-wt for etoposide. The fold change between H69-GLI1 and H69-wt was 2.77 in the drug-dose response experiment in H69-GLI1 versus H69-wt for cisplatin.

The same trend was also observed in combination dosing of cisplatin and etoposide. H526-GLI1 showed no significant change in cisplatin resistance compared to H526-wt. In the four additional SCLC lines a positive trend was observed where increased GLI1 expression correlated with increased cisplatin resistance (FIG. 2). Gene expression analysis showed that there are over 1200 genes significantly differentially-expressed in H69-GLI1 cells as compared to parental cells.

SFRP1 and FOXA2 gene expression were tested in H69-wt and H69-GLI1 cells and found that both genes are over-expressed in H69-GLI1. Further, microRNA 21 (miR-21) was determined to be over-expressed in H69-GLI1 cells as well (SEQ ID NO: 1 5′ UAGCUUAUCAGACUGAUGUUGA 3′).

The primers for GLI1, SFRP1 and FOXA2 qPCR are listed in Table 1.

TABLE 1
Primers for qPCR of selected genes:

| Gene | | | Sequence |
|---|---|---|---|
| Symbol | Primer ID | Primer Sequence (5′→3′) | ID NO. |
| GLI1 | GLI1_F_v1 | ACACCGGTACCACTGTGTCC | 2 |
| | GLI1_R_v1 | CGGCTGACAGTATAGGCAGA | 3 |
| GLI1 | GLI1_F_v2 | AGTACATGCTGGTGGTTCACATGC | 4 |
| | GLI1_R_v2 | AGTATGACTTCCGGCACCCTTCAA | 5 |
| SFRP1 | SFRP1_F | TCTGAGGCCATCATTGAACA | 6 |
| | SFRP1_R | TCAGGGGCTTCTTCTTCTTG | 7 |
| FOXA2 | FOXA2_F | CCGACTGGAGCAGCTACTATG | 8 |
| | FOXA2_R | TGTACGTGTTCATGCCGTTC | 9 |

Further, RNA from H69-wt (wild type) and H69-GLI1 over expressing cells was measured on gene expression array (Agilent) and significant differentially expressed genes were observed. The top candidates including AIM1, ALK, CCDO62, CXCR7, DIRAS2, FAM101A, GRK1, PNMA2, PPP2R2B, RGS20, SOX15 were validated by qPCR, and they are expressed significantly higher in the H69-GLI1 over expressing cells relative to the H69-wt (Table 2 and FIG. 3). The primers used qPCR for these genes are listed in Table 3.

TABLE 2
Differentially expressed gene list:

| | | UniProtKB/ | Log Ratio |
|---|---|---|---|
| | | Swiss-Prot | Relative to |
| Gene Symbol | Protein Name | ID Number | Wild Type |
| AIM1 | Membrane-associated | Q9UMX9 | 2.62 |
| | transporter protein | | |
| ALK | ALK tyrosine kinase | Q9UM73 | 2.31 |
| | receptor | | |
| CCDC62 | Coiled-coil domain- | Q6P9F0 | 3.125 |
| | containing protein 62 | | |
| CXCR7 | C—X—C chemokine | P25106 | 1.185 |
| | receptor type 7 | | |
| DIRAS2 | GTP-binding protein | Q96HU8 | 3.51333333 |
| | Di-Ras2 | | |
| FAM101A | Protein FAM101A | Q6ZTI6 | 10.685 |
| GRK1 | Rhodopsin kinase | Q15835 | 4.81166667 |
| PNMA2 | Paraneoplastic antigen | Q9UL42 | 2.50333333 |
| | Ma2 | | |
| PPP2R2B | Serine/threonine-protein | Q00005 | 1.29 |
| | phosphatase 2A 55 kDa | | |
| | regulatory subunit B | | |
| | beta isoform | | |
| RGS20 | Regulator of G-protein | O76081 | 3.67333333 |
| | signaling 20 | | |
| SOX15 | Protein SOX-15 | O60248 | 1.28666667 |

TABLE 3
Primers for qPCR and amplicon sequences of selected genes:

| | | Primer | Sequence | | Sequence |
|---|---|---|---|---|---|
| Gene | | Sequence | ID | | ID |
| Symbol | Primer ID | (5′→3′) | NO. | Amplicon Sequence (5′→3′) | NO. |
| AIM1 | AIM1_F | CTGGAATGT | 10 | CTGGAATGTCATTATCAGACACAAT | 32 |
| | | CATTATCAG | | GACACTTAGAGGAAGTGTCCAAAA | |
| | | ACACAA | | TAAACTCAATCCCCGACCTGGAAA | |
| | AIM1_R | TCAGAGACG | 11 | GGTAGTGATATATAGTGAACCCGA | |
| | | TCGGGTTCA | | CGTCTCTGA | |
| | | CT | | | |
| ALK | ALK_F | GGGAAGCAT | 12 | GGGAAGCATGGTTGGACAGTGCTC | 33 |
| | | GGTTGGACA | | CAGGGAAGAATCGGGCGTCCAGAC | |
| | ALK_R | ACTCGAAAT | 13 | AACCCATTTCGAGT | |
| | | GGGTTGTCT | | | |
| | | GG | | | |
| CCDC | CCDC62_F | AGGAAACA | 14 | AGGAAACAAAAGGCAACTTCAGTA | 34 |
| | | AAAGGCAA | | TTCATCGTGATCACGAATTTCTCAT | |
| | | CTTCAG | | CTATGTGGAAGGCAGAAAGCAGAC | |
| | CCDC62_R | ATTCATTCA | 15 | ACCAATACTGAATGAAT | |
| | | GTATTGGTG | | | |
| | | TCTGCT | | | |
| CXCR7 | CXCR7_F | CGATGCCTC | 16 | CGATGCCTCCAGAGTCTCAGAGAC | 35 |
| | | CAGAGTCTC | | GGAGTACTCTGCCTTGGAGCAGAG | |
| | | A | | CACCAAATGATCTGCC | |
| | CXCR7_R | GGCAGATCA | 17 | | |
| | | TTTGGTGCT | | | |
| | | CT | | | |
| DIRAS2 | DIRAS2_F | GAAGCTCTG | 18 | GAAGCTCTGAGCGGAGTTGTGTTCT | 36 |
| | | AGCGGAGTT | | TCCCCAGGTGCGTCCTGGCTGAGA | |
| | | GT | | GTTGGAGCTCTCCAGCAACATGCCT | |
| | DIRAS2_R | TCTGCTCAG | 19 | GAGCAGA | |
| | | GCATGTTGC | | | |
| | | T | | | |
| FAM101A | FAM101A_F | CTCAGCCGT | 20 | CTCAGCCGTAGGCGTCTTTGCCCGG | 37 |
| | | AGGCGTCTT | | AGCTGTGAGCCCCCCTCCCAACTCC | |
| | FAM101A_R | TGATGCTCT | 21 | CAAATCCCCCGGCGTCGGAGATGA | |
| | | CCCCAAAGA | | GGCCCCGGATGCTGCCAGTGTTCTT | |
| | | AC | | TGGGGAGAGCATCA | |
| GRK1 | GRK1_F | CCCCTCTTC | 22 | CCCCTCTTCAAGGACCTTAACTGGA | 38 |
| | | AAGGACCTT | | GGCAGCTGGAGGCTGGGATGCTGA | |
| | | AACT | | TGCCCCCTTTCATCCCAGACTCCAA | |
| | GRK1_R | TTGGAGTCT | 23 | | |
| | | GGGATGAA | | | |
| | | AGG | | | |
| HRASLS | HRASLS_F | AGAGACCCC | 24 | AGAGACCCCAGGACACACACAGCT | 39 |
| | | AGGACACAC | | GCCTCCCGGTGCGAGAAGAAGACC | |
| | | AC | | CCGGCTTGAGAGTGAGATGGCGTT | |
| | HRASLS_R | GCAATCATT | 25 | TAATGATTGC | |
| | | AAACGCCAT | | | |
| | | CTC | | | |
| PPP2R2B | PPP2R2B_F | ATCCTGCCA | 26 | ATCCTGCCACCATCACAACCCTGCG | 40 |
| | | CCATCACAA | | GGTGCCTGTCCTGAGACCCATGGA | |
| | | C | | CCTGATGGTGGAGGCCACCCCACG | |
| | PPP2R2B_R | GCGTTGGCA | 27 | AAGAGTATTTGCCAACGC | |
| | | AATACTCTT | | | |
| | | CG | | | |
| RGS20 | RGS20_F | CCTCAGGGC | 28 | CCTCAGGGCTGTTCCTGATATCAAG | 41 |
| | | TGTTCCTGA | | TCCTTCCCGCCTGCACAGCTCCCAG | |
| | | TA | | ACTCGCCCGCCGCCCCGAAGCTGTT | |
| | RGS20_R | GCTAGAAAG | 29 | CGGCCTCCTTTCTAGC | |
| | | GAGGCCGA | | | |
| | | AC | | | |
| SOX15 | SOX15_F | AAGTTCCTC | 30 | AAGTTCCTCGGCAACGACTCCAGA | 42 |
| | | GGCAACGAC | | CTGGGAAGACCTTTCCATTTTCAGG | |
| | | T | | ATCGACGCTTC | |
| | SOX15_R | GAAGCGTCG | 31 | | |
| | | ATCCTGAAA | | | |
| | | AT | | | |

These validated candidate genes were entered in Ingenuity Pathway Analysis (IPA, Ingenuity Systems, Inc Redwood City, Calif.) to look for significantly altered pathways by one or more of these candidate genes. The pathway analysis demonstrates that genes upregulated by GLI1 overexpression can stimulate and/or activate Wnt and cell cycle regulation pathways. In particular, PPP2 (of which PPP2R2B is a subunit) and SOX15 were found to affect significantly the Wnt/β-caternin signaling pathway by inhibiting Wnt/β-caternin signaling, likely, due to reduction in nuclear β-catenin (Akiyoshi et al., (2006) Gut. 55(7):991-9). Further, PPP2 also affects the cyclins and cell cycle regulation pathways by activating the pathways. These pathways may be implicated to contribute to mechanism of chemoresistance when GLI1 is upregulated.

What is claimed is:

1. A biomarker profile, the profile comprises one or more biomarkers selected from the group consisting of GLI1, SFRP1, FOXA2, MIR21, PPP2R2B and SOX15.

2. The biomarker profile of claim 1, wherein the profile is for identifying chemotherapeutic drug resistance in a sample from a subject.

3. The biomarker profile of claim 2, wherein the sample comprises at least one cancer cell.

4. The biomarker profile of claim 3, wherein the cancer cell is a Small Cell Lung Cancer (SCLC) cell.

5. The biomarker profile of claim 2, wherein the subject is a mammal.

6. The biomarker profile of claim 5, wherein the mammal is a human.

7. The biomarker profile of claim 6, wherein the human is known or diagnosed as having chemotherapeutic drug resistant cancer.

8. The biomarker profile of claim 1, wherein the one or more biomarkers selected from the group consisting of GLI1, SFRP1, FOXA2, MIR21, PPP2R2B and SOX15 is overexpressed in cancer cells resistant to a chemotherapy drug relative to their respective control level of expression in a cancer cell responsive to the chemotherapeutic drug.

9. The biomarker profile of claim 8, wherein the chemotherapeutic drug is cisplatin or etoposide.

10. A method for identifying chemotherapeutic drug resistance in a sample from a subject, comprising:
a. receiving the sample;
b. detecting overexpression of one or more biomarkers selected from the group consisting of GLI1, SFRP1, FOXA2, MIR21, PPP2R2B and SOX15 in the sample relative to their respective control level of expression in a cancer cell responsive to the chemotherapeutic drug; and
c. identifying chemotherapeutic drug resistance as being present in a sample having overexpression of one or more of said biomarkers.

11. The method of claim 10, wherein the sample comprises at least one cancer cell.

12. The method of claim 11, wherein the cancer cell is a SCLC cell.

13. The method of claim 10, wherein the subject is a mammal.

14. The method of claim 13, wherein the subject is a human.

15. The method of claim 14, wherein the human is known or diagnosed as having chemotherapeutic drug resistant cancer.

16. The method of claim 10, wherein the overexpression of the one or more biomarkers is determined in a form selected from the group consisting of a nucleic acid, protein, peptide, and a fragment thereof.

17. The method of claim 10, wherein the overexpression of the one or more biomarkers is determined by a technique selected from the group consisting of quantitative real-time PCR, microarray, Western blotting, ELISA, and immunohistochemistry.

18. A kit for detecting altered expression of one or more biomarkers associated with cancer cell resistance to a chemotherapeutic drug, comprising:
one or more reagents for detecting at least one biomarker selected from the group consisting of GLI1, SFRP1, FOXA2, MIR21, PPP2R2B and SOX15.

19. The kit of claim 18, wherein the one or more reagents comprise one or more primers, probes, or antibodies that recognize a specific biomarker selected from the group consisting of GLI1, SFRP1, FOXA2, MIR21, PPP2R2B and SOX15.

20. The kit of claim 19, wherein the chemotherapeutic drug is cisplatin or etoposide.
